(19) United States
(12) Patent Application Publication
(10) Pub. No.: US 2024/0081718 A1
(43) Pub. Date: Mar. 14, 2024

(54) BIOLOGICAL INFORMATION MEASUREMENT DEVICE

(71) Applicant: OMRON HEALTHCARE Co., Ltd., Kyoto (JP)

(72) Inventors: Kenji FUJII, Kyoto (JP); Yasuhiro KAWABATA, Kyoto (JP); Naomi MATSUMURA, Kyoto (JP); Akito ITO, Kyoto (JP); Yuki SAKAGUCHI, Kyoto (JP); Daizo OKA, Kyoto (JP); Takuya YOSHIDA, Kyoto (JP); Seiji FUKUNAGA, Kyoto (JP); Tatsuaki OKA, Kyoto (JP)

(21) Appl. No.: 18/511,478

(22) Filed: Nov. 16, 2023

Related U.S. Application Data
(63) Continuation of application No. PCT/JP2021/026694, filed on Jul. 15, 2021.

(51) Int. Cl. A61B 5/364; A61B 5/00; A61B 5/30

(52) U.S. Cl. CPC A61B 5/364; A61B 5/30; A61B 5/7225; A61B 5/74; A61B 5/6801; A61B 2560/0462

(57) ABSTRACT

A biological information measurement device includes a sensor unit that detects predetermined biological information related to an organ of a living body, an A/D conversion unit that converts a measurement signal output from the sensor unit into a digital signal, a storage unit that stores information including a digital signal related to the measurement signal output from the A/D conversion unit, an analysis processing unit that determines presence or absence of a suspicion of an abnormality in the organ by analyzing the digital signal, and a measurement control unit that changes a sampling frequency related to A/D conversion of the measurement signal under a predetermined condition when the analysis processing unit has determined that a suspicion of an abnormality is present in the organ.

### CROSS-REFERENCE TO RELATED APPLICATIONS

This application is the U.S. national stage application filed pursuant to 35 U.S.C. 365(c) and 120 as a continuation of International Patent Application No. PCT/JP2021/026694, filed Jul. 15, 2021, which application is incorporated herein by reference in its entirety.

### TECHNICAL FIELD

The present invention belongs to the technical field related to healthcare, and particularly relates to a biological information measurement device.

### BACKGROUND ART

In recent years, health management has been becoming more and more common in which information related to individual bodies and health (hereinafter, also referred to as biological information), such as a blood pressure value and an electrocardiographic waveform, is measured using a measurement device, with the measurement results recorded and analyzed by an information processing terminal.

As an example of a measurement device described above, a biological information measurement device configured to be able to change a sampling period of biological information detection in accordance with a diagnostic purpose has been proposed (Patent Literature 1). Patent Literature 1 describes a biological information measurement device provided with a measurement unit including a photoplethysmograph and an analysis unit, in which a sampling period of sensing by the photoplethysmograph is changed according to a connection state between the measurement unit and the analysis unit. According to such a configuration, it is possible to acquire biological information at different sampling periods that match the purpose of measurement (diagnosis) by one measurement device.

In recent years, there has been an increasing need for early discovery of diseases and appropriate treatment by continuously acquiring biological information with a measurement device constantly attached to the body in daily life. In the case of a so-called wearable device that satisfies such needs, it is desirable that the wearable device be made as small as possible from the viewpoint of wearability, and there are usually restrictions on battery capacity, memory capacity, and the like. For this reason, it is desirable that the data to be constantly measured and recorded be the minimum necessary data, and that data necessary and sufficient for diagnosis be measured and recorded when an abnormality occurs.

In this regard, with the technique of Patent Literature 1, it is possible to change the sampling period of measurement data by connecting the measurement unit and the analysis unit, and it is possible to perform measurement with a long sampling period (low sampling frequency) at the time of normal measurement and acquire data with a short sampling period (high sampling frequency) by connecting the analysis unit at the time of analysis.

### CITATION LIST

### Patent Literature

Patent Literature 1: JP 2007-117586 A

### SUMMARY OF INVENTION

### Technical Problem

However, with the technique described in the above Patent Literature 1, in order to change the sampling frequency, it is necessary to perform operations, such as connecting the connection unit (pressing an analysis button), and it is necessary to manually switch the sampling period. For this reason, there has been a problem that it takes time to switch the sampling period each time. In addition, since the user needs to switch the sampling period manually with awareness, it is difficult to capture an abnormality (or a sign thereof), such as atrial fibrillation of the heart, and switch the sampling period at an appropriate timing.

In view of the problems described above, an object of the present invention is to provide a technique that allows determining presence or absence of a suspicion of an abnormality in an organ as a measurement target based on biological information measured by a biological information measurement device and automatically switching a sampling frequency of measurement data according to the determination result.

### Solution to Problem

The biological information measurement device according to the present invention adopts the following configurations to solve the above-described problems. That is, a biological information measurement device includes:
a sensor unit that detects predetermined biological information related to an organ of a living body;
an A/D conversion unit that converts a measurement signal output from the sensor unit into a digital signal;
a storage unit that stores information including a digital signal related to the measurement signal output from the A/D conversion unit;
an analysis processing unit that determines presence or absence of a suspicion of an abnormality in the organ by analyzing the digital signal; and
a measurement control unit that changes a sampling frequency related to A/D conversion of the measurement signal under a predetermined condition when the analysis processing unit has determined that a suspicion of an abnormality is present in the organ.

According to such a configuration, for example, it is possible to automatically change the sampling frequency in the A/D conversion unit between a normal time and a state in which there is a suspicion of an abnormality is present (hereinafter, also simply referred to as an abnormal time). Thus, for example, it is possible to provide a biological information measurement device that can perform continuous measurement for a long period by suppressing power consumption at a low sampling frequency at a normal time, and switch to measurement at a high sampling frequency so as to automatically acquire data required for diagnosis at an abnormal time.

In addition, the analysis processing unit may determine that a suspicion of an abnormality is present in the organ when the digital signal satisfies a predetermined second condition, and the measurement control unit may change the sampling frequency from a predetermined first frequency to a predetermined second frequency set to have a value higher than the first frequency over a predetermined period of time set in advance when the analysis processing unit has determined that a suspicion of an abnormality is present in the organ. The second condition referred to here can be, for example, that a predetermined index related to the biological information deviates from a predetermined threshold. According to such a configuration, when an abnormality is suspected, it is possible to acquire biological information for a necessary and sufficient time at a high sampling frequency set to obtain necessary and sufficient data for performing diagnosis.

In addition, the analysis processing unit may determine that a suspicion of an abnormality is present in the organ when the digital signal satisfies a predetermined second condition, and the measurement control unit may change the sampling frequency from a predetermined first frequency to a predetermined second frequency set to have a value higher than the first frequency while the second condition is satisfied. With such a configuration, it is possible to continuously acquire biological information at a high sampling frequency set to obtain data necessary and sufficient for performing diagnosis as long as the suspicion of an abnormality continues.

In addition, the organ may be a heart, and the measurement signal may be an electrocardiographic signal. When the analysis processing unit has not determined that a suspicion of an abnormality is present in the heart, a heartbeat interval obtained from the digital signal sampled at the first frequency may be stored in the storage unit, and when the analysis processing unit has determined that a suspicion of an abnormality is present in the heart, an electrocardiographic waveform obtained from the digital signal sampled at the second frequency may be stored in the storage unit. Here, the heartbeat interval can be, for example, an R-R interval of a waveform that can be acquired from the electrocardiographic signal. With such a configuration, the contents of the data stored in the storage unit can be automatically switched between the normal time and the abnormal time without requiring operation by the user. Specifically, for example, at a normal time, the heartbeat interval can be continuously stored based on data of a low sampling frequency, and data can be continuously deleted in order from the oldest piece of data while leaving only an amount of data required for determining the presence or absence of an abnormality (that is, information required for determining the presence or absence of an abnormality is temporarily stored). On the other hand, when an abnormality is suspected, the data acquired at the high sampling frequency, specifically, the electrocardiographic waveform having the necessary and sufficient quality and quantity for diagnosis can be stored until it is intentionally deleted (that is, non-temporarily). According to this, it is possible to non-temporarily store only the data acquired at the high sampling frequency at an abnormal time in the storage unit, and it is possible to save the storage capacity.

In addition, the organ may be a heart, the measurement signal may be an electrocardiographic signal, and the analysis processing unit may determine presence or absence of a suspicion of an abnormality in the heart based on a heartbeat interval calculated based on the digital signal. For example, an R-R interval of a waveform that can be acquired from an electrocardiographic signal can be detected as a heartbeat interval and temporarily stored, and the presence or absence of a suspicion of an abnormality in the heart can be determined based on a variation in the heartbeat intervals.

In addition, the analysis processing unit may determine that the suspicion of an abnormality is present when a variation value of the heartbeat intervals deviates from a predetermined threshold. That is, the above-described second condition may be that “a variation value of the heartbeat intervals deviates from a predetermined threshold”. With such a configuration, it is possible to easily and reliably determine the presence or absence of a suspicion of an abnormality.

Further, the biological information measurement device may be a wearable device configured to be allowed to constantly be attached to the living body. The present invention is suitable for such a device having a large restriction on battery capacity and storage capacity.

In addition, the analysis processing unit may further include notification means for notifying information thereon when the analysis processing unit has determined that a suspicion of an abnormality is present in the organ. With such a configuration, when a suspicion of an abnormality is present, the user can be aware of the fact, and can take a desirable action for acquiring biological information at an abnormal time, such as take a resting posture.

Also, the configurations and processing described above can be combined with one another to constitute the present invention unless the combination leads to contradiction.

### Advantageous Effects of Invention

With the present invention, it is possible to provide a technique that allows determining presence or absence of a suspicion of an abnormality in an organ as a measurement target based on biological information measured by a biological information measurement device, and automatically switching a sampling frequency of measurement data according to the determination result.

### BRIEF DESCRIPTION OF DRAWINGS

FIG. 1A is an external perspective view illustrating an overview of a wearable electrocardiograph according to an embodiment of the present invention. FIG. 1B is a front view illustrating the overview of the wearable electrocardiograph according to the embodiment of the present invention.

FIG. 2 is a block diagram illustrating a functional configuration of the wearable electrocardiograph according to the embodiment.

FIG. 3 is a flowchart illustrating an example of electrocardiographic measurement processing in the wearable electrocardiograph according to the embodiment.

FIG. 4 is a flowchart illustrating a flow of a subroutine in the electrocardiographic measurement processing in the wearable electrocardiograph according to the embodiment.

FIG. 5A is a first explanatory diagram indicating a relationship between an electrocardiographic waveform and heartbeat intervals. FIG. 5B is a second explanatory diagram indicating an electrocardiographic waveform and heartbeat intervals.

FIG. 6 is a flowchart illustrating a flow of the electrocardiographic measurement processing according to a modified example of the embodiment.

### DESCRIPTION OF EMBODIMENTS

### First Embodiment

Embodiments of the present invention will be specifically described below with reference to the drawings. It should be noted that the dimension, material, shape, relative arrangement and the like of the configurations described in this embodiment are not intended to limit the scope of this invention to them alone, unless otherwise stated.

Electrocardiographic Waveform Measurement Device

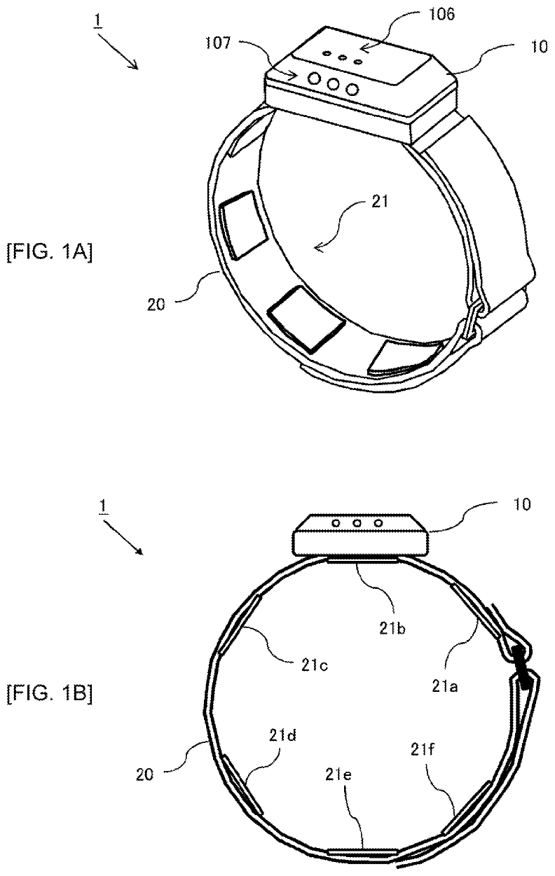

FIG. 1A and FIG. 1B are schematic diagram illustrating a configuration of a wearable electrocardiograph 1 according to the present embodiment, in which FIG. 1A is an external perspective view of the wearable electrocardiograph 1, and FIG. 1B is a front view of the wearable electrocardiograph 1.

As illustrated in FIG. 1A and FIG. 1B, the wearable electrocardiograph 1 generally includes a main body portion 10 including a control unit (not illustrated in FIG. 1A and FIG. 1B), an operation unit 107, a display unit 106, and the like, and a belt portion 20 including an electrode unit 21 constituted of a plurality of electrodes 21a, 21b, 21c, 21d, 21e, and 21f. Each electrode of the electrode unit 21 is electrically connected to the main body portion 10 via a conductive wire (not illustrated) or the like disposed inside the belt portion 20. The user wears the wearable electrocardiograph 1 on, for example, the left upper arm using the belt portion 20 such that each electrode of the electrode unit 21 comes into contact with the skin surface, thereby enabling continuous electrocardiographic measurement at all times.

The operation unit 107 is constituted of a plurality of operation buttons (such as a selection button, a determination button, and a power button). The display unit 106 is configured as an indicator (such as abnormality notification, communication state display, and battery state display) by a plurality of LEDs, for example.

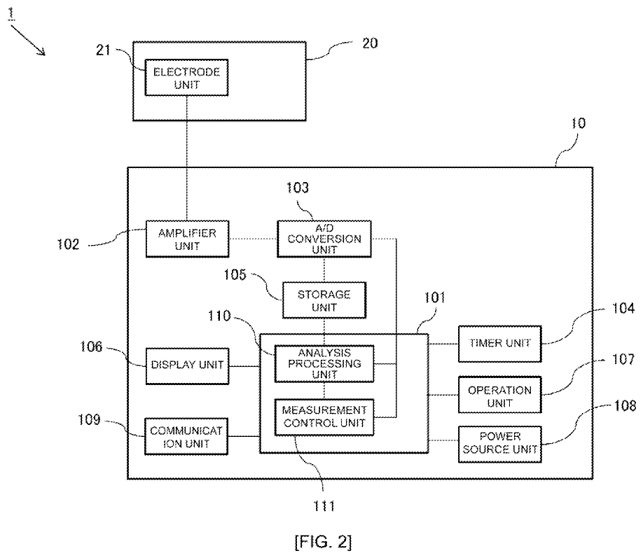

FIG. 2 illustrates a block diagram illustrating a functional configuration of the wearable electrocardiograph 1. As illustrated in FIG. 2, the wearable electrocardiograph 1 includes respective function units including a control unit 101, the electrode unit 21, an amplifier unit 102, an analog to digital (A/D) conversion unit 103, a storage unit 105, the display unit 106, the operation unit 107, a power source unit 108, a communication unit 109, an analysis processing unit 110, and a measurement control unit 111.

The control unit 101 is means for controlling the wearable electrocardiograph 1, and is configured including a central processing unit (CPU) and the like. In response to receiving operation of the user via the operation unit 107, the control unit 101 controls each component of the wearable electrocardiograph 1 to execute various processing, such as electrocardiographic measurement and information communication, in accordance with a predetermined recording medium. Note that the predetermined recording medium is stored in the storage unit 105 described below and is read out from here. Additionally, the control unit 101 includes, as a functional module, the analysis processing unit 110 for analyzing electrocardiographic signals and the measurement control unit 111. These function units will be described in detail below.

The electrode unit 21 includes the six electrodes 21a, 21b, 21c, 21d, 21e, and 21f, and functions as a sensor unit for detecting electrocardiographic signals. Specifically, in a state in which the wearable electrocardiograph 1 is worn, two electrodes in an opposing positional relationship form a pair, and an electrocardiographic signal is detected based on a potential difference between the two electrodes forming the pair. That is, three kinds of electrocardiographic signals can be simultaneously detected from the three pairs of electrodes. The amplifier unit 102 has a function of amplifying signals output from the electrode unit 21.

The A/D conversion unit 103 converts analog signals amplified by the amplifier unit 102 into digital signals at a predetermined sampling frequency under the control of the measurement control unit 111 and outputs the digital signals. The output signals are processed under the control of the measurement control unit 111 and stored in the storage unit 105. As will be described in detail below, a sampling frequency in the A/D conversion unit 103 and the content of the information stored in the storage unit 105 can be changed under the control of the measurement control unit 111.

A timer unit 104 functions to measure time with reference to a real time clock (RTC, not illustrated). For example, as will be described below, the time when a predetermined event occurs is counted and output.

The storage unit 105 includes a main memory device (not illustrated), such as a random access memory (RAM), and stores various types of information, such as application recording mediums and data transmitted from the A/D conversion unit 103 (heartbeat information and electrocardiographic waveforms). In addition to the RAM, for example, a long-term storage medium, such as a flash memory, is provided.

The display unit 106 includes light-emitting elements, such as LEDs, and informs the user of the state of the device and the occurrence of a predetermined event by lighting, blinking, or the like of the LEDs. Additionally, the operation unit 107 includes a plurality of operation buttons, and functions to receive an input operation from the user via the operation buttons and to cause the control unit 101 to execute processing in accordance with the operation.

The power source unit 108 includes a battery (not illustrated) that supplies power required for operation of the device. The battery may be, for example, a secondary battery, such as a lithium ion battery, or a primary battery. In the case of including a secondary battery, a charging terminal or the like may be provided. The communication unit 109 includes, for example, an antenna for wireless communication and a wired communication terminal (both unillustrated), and functions to communicate with another device, such as an information processing terminal. Note that the communication unit 109 may also serve as a charging terminal.

The analysis processing unit 110 analyzes data stored in the storage unit 105, determines whether or not a suspicion of an abnormality is present in the heart (or the behavior thereof) based on the heartbeat intervals obtained from the data, and outputs the result. Specifically, for example, when a variation value of the heartbeat intervals deviates from predetermined thresholds (upper and lower limit values), it is determined that a suspicion of an abnormality is present in the heart.

The measurement control unit 111 controls the sampling frequency of the A/D conversion unit 103 and the content of the data stored in the storage unit 105 based on a predetermined condition. To be more specific, at a normal time when no suspicion of an abnormality is present in the heart (when the analysis processing unit 110 has not output a determination result of an abnormality), control is performed such that the electrocardiographic signals are digitally converted (sampled) at a low sampling frequency for a normal time (for example, from 30 Hz to 50 Hz), heartbeat intervals are extracted from a waveform of the signals (hereinafter, information related to the heartbeat intervals is also referred to as heartbeat interval data), and the heartbeat interval data is stored in the storage unit 105. Hereinafter, the sampling frequency at the normal time is also simply referred to as a low frequency. The heartbeat interval can be obtained by, for example, extracting peaks of amplitude (corresponding to R waves of an electrocardiogram) in the electrocardiographic waveform and obtaining a time interval between adjacent peaks. The storage of the heartbeat interval data in the storage unit 105 is temporary, and the oldest piece of data is constantly deleted while leaving (coherent) heartbeat interval data required for the analysis processing unit 110 to perform abnormality determination.

On the other hand, at an abnormal time at which a suspicion of an abnormality is present in the heart (when the analysis processing unit 110 has output a determination result of an abnormality), the measurement control unit 111 changes the sampling frequency to a value (for example, from 250 Hz to 1000 Hz) high enough to obtain an electrocardiographic waveform that can be used as an electrocardiogram. Hereinafter, the sampling frequency at an abnormal time is also simply referred to as a high frequency. Thereafter, when a predetermined condition is satisfied, the sampling frequency is changed to a low frequency again. Note that waveform data (hereinafter, referred to as electrocardiographic waveform data) obtained from electrocardiographic signals digitally converted at a high frequency at an abnormal time is stored in the storage unit 105 as non-temporary data.

Electrocardiographic Measurement Processing Using Wearable Electrocardiograph

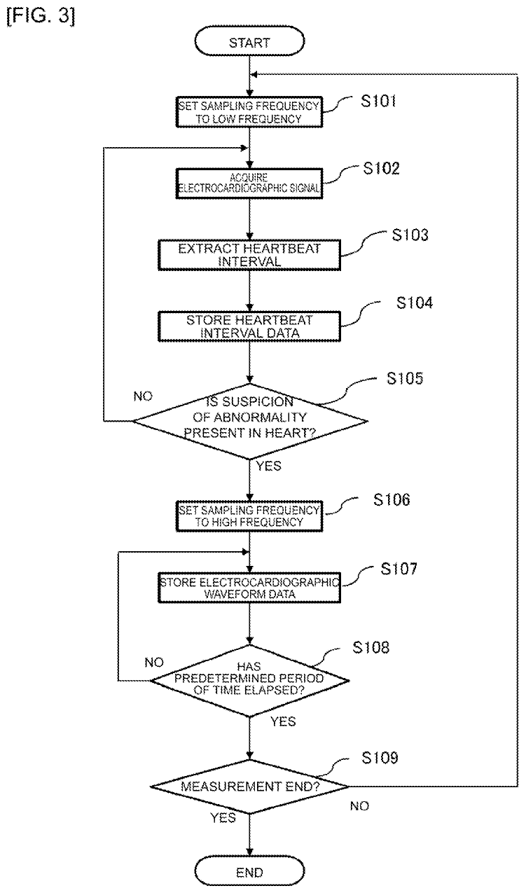

Next, based on FIG. 3, a description will be given for operation of the wearable electrocardiograph 1 that is performed when the electrocardiographic measurement is performed. FIG. 3 is a flowchart illustrating a procedure of processing executed when the electrocardiographic measurement is performed using the wearable electrocardiograph 1 according to the present embodiment.

Prior to the electrocardiographic measurement, the user wears the wearable electrocardiograph 1 on, for example, the left upper arm using the belt portion 20 such that each electrode of the electrode unit 21 comes into contact with the skin surface. By operating the operation buttons, the electrocardiographic measurement is started.

When the electrocardiographic measurement is started, the control unit 101 (measurement control unit 111) first sets the sampling frequency of the A/D conversion unit 103 to a low frequency (S101). Then, electrocardiographic signals are acquired from the electrode unit 21 (S102) and digitally converted at a low frequency by the A/D conversion unit 103, heartbeat intervals are extracted from the waveform of the signals (S103), and heartbeat interval data is stored in the storage unit 105 (S104). Subsequently, the analysis processing unit 110 performs an abnormality presence/absence determination of whether or not a suspicion of an abnormality is present in the heart (S105).

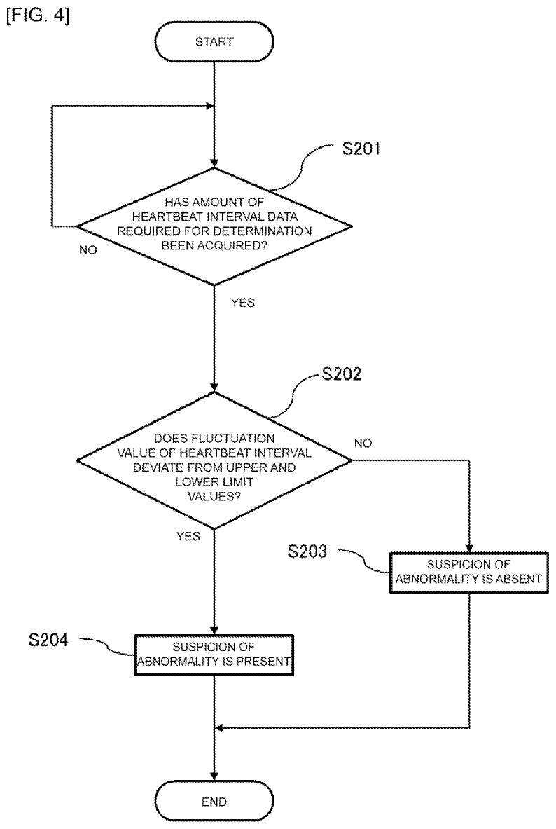

FIG. 4 illustrates a flow of a subroutine of the abnormality presence/absence determination processing performed in step S105. As illustrated in FIG. 4, first, the analysis processing unit 110 checks whether an amount of heartbeat interval data required for the presence/absence determination of abnormality is stored in the storage unit 105 (S201). Here, when determined that a required amount of heartbeat interval data is not stored, the processing of step S201 is repeated. On the other hand, when it is determined that the required amount of data is stored, it is determined whether or not a variation value of the heartbeat intervals deviates from predetermined upper and lower limit thresholds based on the data (S202).

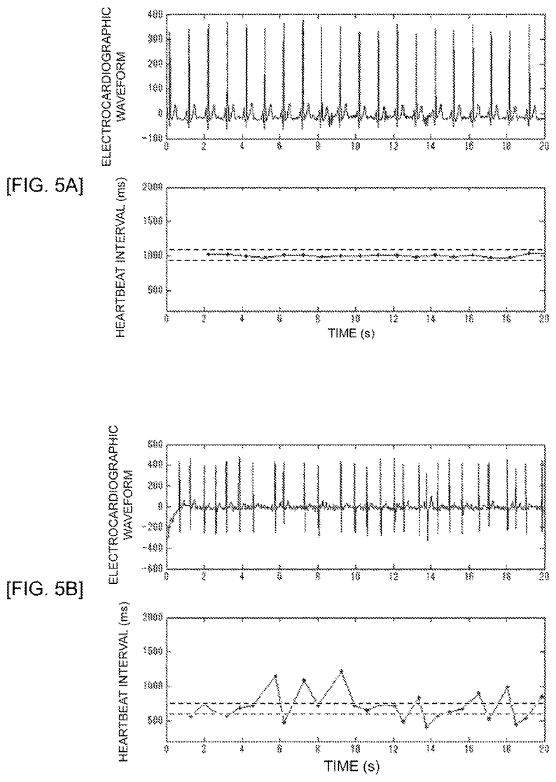

FIG. 5A and FIG. 5B are graph indicating heartbeat interval data at a normal time without abnormality and heartbeat interval data at an abnormal time. In FIG. 5A, the heartbeat interval data at the normal time is indicated as a graph with time on the X-axis and the value of the heartbeat interval on the Y-axis, together with a corresponding graph of an electrocardiographic waveform. In FIG. 5B, the heartbeat interval data at the abnormal time is indicated as a graph with time on the X-axis and the value of the heartbeat interval on the Y-axis, together with a corresponding graph of an electrocardiographic waveform. Broken lines in the figure indicate upper and lower limit thresholds for abnormality presence/absence determination, and the thresholds can be, for example, values of ±25 ms of the average heartbeat interval.

In step S202, when a variation value of the heartbeat intervals does not deviate from the upper and lower limit thresholds, the analysis processing unit 110 determines that the heart (the behavior thereof) is normal (S203), and the subroutine is ended. On the other hand, when the heartbeat intervals deviate from the upper and lower limit thresholds, it is determined that a suspicion of an abnormality is present in the heart (S204), and the subroutine is ended.

Returning to the description of the flow of the entire electrocardiographic measurement illustrated in FIG. 3, when it is determined in step S105 that no suspicion of an abnormality is present in the heart (normal), the flow returns to step S102 and the subsequent processing is repeated. On the other hand, when it is determined in step S105 that a suspicion of an abnormality is present, the measurement control unit 111 changes the sampling frequency in the A/D conversion unit 103 to a high frequency (S106). Subsequently, the signals sampled at the high frequency are stored in the storage unit 105 as electrocardiographic waveform data for an electrocardiogram (S107).

Thereafter, the measurement control unit 111 refers to the timer unit 104 and determines whether or not a predetermined period of time (for example, seconds) has elapsed (S108). Here, when it is determined that the predetermined period of time has not elapsed, the flow returns to step S107, and the subsequent processing is repeated. On the other hand, when it is determined in Step S108 that the predetermined period of time has elapsed, the flow proceeds to Step S109, and it is determined whether or not conditions for ending the measurement (such as, the end button having been pressed or a sufficient amount of storage capacity not being left) are satisfied (S109). Here, when it is determined that conditions for ending the measurement are not met, the flow returns to step S101, and the subsequent processing is repeated. On the other hand, when it is determined in step S109 that the conditions for ending the measurement are satisfied, the measurement is ended.

According to the wearable electrocardiograph 1 as described above, it is possible to automatically perform processing of acquiring only heartbeat interval data required for determining the presence or absence of an abnormality at a low frequency at a normal time, and acquiring electrocardiographic waveform data usable for diagnosis at a high frequency and non-temporarily storing the electrocardiographic waveform data when there has occurred a suspicion of an abnormality. For this reason, it is possible to provide a wearable electrocardiograph in which trouble of switching the sampling frequency is eliminated, and when a suspicion of an abnormality is present, the sampling frequency is changed at an appropriate time and data required for diagnosis is stored. Accordingly, even when the wearable device is limited in power source (battery capacity) or storage capacity, it is possible to increase the possibility of capturing an abnormality in the heart by performing continuous measurement for a long time.

### Modified Example

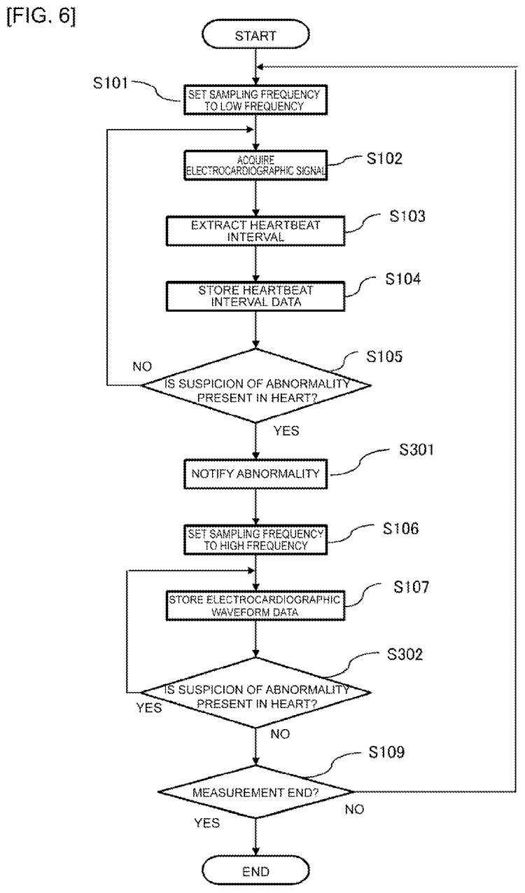

Note that the above-described configuration and processing can be appropriately changed. For example, in the above-described first embodiment, the acquisition of electrocardiographic waveform data at the abnormal time is ended after the elapse of a predetermined period of time. However, the timing at which the acquisition of the electrocardiographic waveform data is ended may be determined by a method other than this. FIG. 6 illustrates a flowchart of electrocardiographic measurement processing according to such a modified example. Note that, in the modified example, processing similar to the first embodiment described above is denoted with the same reference numerals and a detailed description thereof is omitted.

Even in the processing of the modified example, the flow is substantially similar to that of the electrocardiographic measurement processing of the first embodiment. That is, the measurement is started, a sampling frequency is set to a low frequency (S101), electrocardiographic signals are acquired (S102), heartbeat intervals are extracted from the electrocardiographic signals (S103), heartbeat interval data is stored (S104), and abnormality determination processing of the heart is performed (S105) (please refer to FIG. 3).

Here, when it is determined that a suspicion of an abnormality is present in the heart, in the present modified example, processing of notifying the user of the possibility of the abnormality is continuously performed (S301). Specifically, for example, notification may be performed by lighting or blinking the LEDs of the display unit 106, or notification may be performed by sound with a configuration including a buzzer or the like. In this way, the user can take measures desirable for accurate measurement of the electrocardiographic waveform, such as maintaining a resting state.

The control unit 101 performs the processing of step S301, changes the sampling frequency to a high frequency (S106), and stores electrocardiographic waveform data in the storage unit 105 (S107). Then, the analysis processing unit 110 performs abnormality presence/absence determination processing of the heart based on the electrocardiographic waveform data (S302). The processing performed in the determination processing in step S302 is similar to the processing of the subroutine in step S105. The heartbeat interval data can naturally be acquired from the digital signals sampled at a high frequency.

When it is determined in step S302 that a suspicion of an abnormality is present, the flow returns to step S107 and the subsequent processing is repeated. On the other hand, when is determined as normal in step S302, the flow returns to step S109. The subsequent processing is similar to that in the first embodiment.

With the modified example as described above, when a suspicion of an abnormality is present, the user can be aware of it, and the electrocardiographic waveform data can be stored without interruption as long as the suspicion of an abnormality continues.

Other Points

The description of each example described above is merely illustrative of the present invention, and the present invention is not limited to the specific embodiments described above. Within the scope of the technical idea of the present invention, various modifications and combinations can be made. For example, in the above-described embodiment, the display unit 106 is constituted of an LED indicator, but may include a liquid crystal screen or the like, or may serve as a touch panel display that serves as the operation unit 107 and the display unit. Conversely, the display unit and the operation unit need not be provided.

Moreover, although the above-described electrocardiographic measurement device is of a wearable type, the present invention is also applicable to devices other than the wearable type. Further, the present invention is also applicable to a biological information measurement device (for example, a pulse wave measurement device) other than the electrocardiographic measurement device.

### REFERENCE NUMERALS LIST

1 Wearable electrocardiograph
10 Main body portion
10 Control unit
102 Amplifier unit
103 A/D conversion unit
104 Timer unit
105 Storage unit
106 Display unit
107 Operation unit
108 Power source unit
109 Communication unit
110 Analysis processing unit
111 Measurement control unit
20 Belt portion
21a, 21b, 21c, 21d, 21e, 21f Electrode

1. A biological information measurement device, comprising:
a sensor unit that detects predetermined biological information related to an organ of a living body;
an A/D conversion unit that converts a measurement signal output from the sensor unit into a digital signal;
a storage unit that stores information including a digital signal related to the measurement signal output from the A/D conversion unit;
an analysis processing unit that determines presence or absence of a suspicion of an abnormality in the organ by analyzing the digital signal; and
a measurement control unit that changes a sampling frequency related to A/D conversion of the measurement signal under a predetermined condition when the analysis processing unit has determined that a suspicion of an abnormality is present in the organ, wherein the organ is a heart and the measurement signal is an electrocardiographic signal;
the analysis processing unit determines that a suspicion of an abnormality is present in the heart when the digital signal satisfies a predetermined second condition;
the measurement control unit performs control of:
changing the sampling frequency from a predetermined first frequency to a predetermined second frequency set to have a value higher than the first frequency over a predetermined period of time set in advance or while the second condition is satisfied when the analysis processing unit has determined that a suspicion of an abnormality is present in the heart; and
storing a heartbeat interval obtained from the digital signal sampled at the first frequency in the storage unit when the analysis processing unit has not determined that a suspicion of an abnormality is present in the heart, and storing an electrocardiographic waveform obtained from the digital signal sampled at the second frequency in the storage unit when the analysis processing unit has determined that a suspicion of an abnormality is present in the heart; and,
regarding data of the heartbeat interval, oldest piece of data of the heartbeat interval is constantly deleted from the storage unit while leaving an amount of latest data of the heartbeat interval required for the analysis processing unit to perform the determination of a suspicion of an abnormality, and data of the electrocardiographic waveform is stored non-temporarily in the storage unit.

2. The biological information measurement device according to claim 1, wherein
the analysis processing unit determines presence or absence of a suspicion of an abnormality in the heart based on a heartbeat interval calculated based on the digital signal.

3. The biological information measurement device according to claim 2, wherein
the second condition is that a variation value of the heartbeat intervals deviates from a predetermined threshold.

4. The biological information measurement device according to claim 1, wherein
the biological information measurement device is a wearable device configured to be allowed to constantly be attached to the living body.

5. The biological information measurement device according to claim 2, wherein
the biological information measurement device is a wearable device configured to be allowed to constantly be attached to the living body.

6. The biological information measurement device according to claim 3, wherein
the biological information measurement device is a wearable device configured to be allowed to constantly be attached to the living body.

7. The biological information measurement device according to claim 1 wherein
the analysis processing unit further includes a notification apparatus adapted to notifying information thereon when the analysis processing unit has determined that a suspicion of an abnormality is present in the organ.

8. The biological information measurement device according to claim 2 wherein
the analysis processing unit further includes the notification apparatus adapted to notifying information thereon when the analysis processing unit has determined that a suspicion of an abnormality is present in the organ.

9. The biological information measurement device according to claim 3 wherein
the analysis processing unit further includes the notification apparatus adapted to notifying information thereon when the analysis processing unit has determined that a suspicion of an abnormality is present in the organ.

10. The biological information measurement device according to claim 4 wherein
the analysis processing unit further includes the notification apparatus adapted to notifying information thereon when the analysis processing unit has determined that a suspicion of an abnormality is present in the organ.

11. The biological information measurement device according to claim 5 wherein
the analysis processing unit further includes the notification apparatus adapted to notifying information thereon when the analysis processing unit has determined that a suspicion of an abnormality is present in the organ.

12. The biological information measurement device according to claim 6 wherein
the analysis processing unit further includes the notification apparatus adapted to notifying information thereon when the analysis processing unit has determined that a suspicion of an abnormality is present in the organ.
